(19) United States
(12) Patent Application Publication
(10) Pub. No.: US 2010/0075422 A1
(43) Pub. Date: Mar. 25, 2010

(54) TRANSFECTION MICROARRAYS

(75) Inventors: Wolfgang Bielke, Koln (DE); Jie Kang, Mettmann (DE); Karsten Reihs, Koln (DE)

Correspondence Address: Baker Donelson Bearman, Caldwell & Berkowitz, PC, 555 Eleventh Street, NW, Sixth Floor, Washington, DC, 20004, US

(73) Assignee: QIAGEN GMBH, Hilden (DE)

(21) Appl. No.: 12/517,356

(22) PCT Filed: Dec. 13, 2007
(86) PCT No.: PCT/EP07/10961

§ 371 Date: Sep. 3, 2009

Related U.S. Application Data
(60) Provisional application No. 60/869,775, filed on Dec. 13, 2006.

(51) Int. Cl. C12N 15/10; C40B 40/06

(52) U.S. Cl. 435/455; 506/16

(57) ABSTRACT

A transfection method of introducing sample molecules into cells is provided, comprising:
(a) using a substrate comprising at least one sample spot area which is surrounded by an hydrophobic area;
(b) wherein sample molecules are applied to said at least one sample spot area, thereby placing said sample molecules in the discrete location of said sample spot area;
(c) applying cells to be transfected onto the substrate and incubation under appropriate conditions for entry of the sample molecules into said cells;
   (d) whereby at least a portion of said sample molecules are introduced into said cells.

### CROSS REFERENCE TO RELATED APPLICATIONS

This application is a §371 National Stage Application of PCT/EP07/10961, filed Dec. 13, 2007, which claims priority to U.S. Provisional Application 60/869,775, filed Dec. 13, 2006.

### FIELD OF THE INVENTION

A design proposal is presented for an hydrophobic optical cell array, enabling the transfection of defined sample molecules, such as e.g. siRNAs, into cells. The present invention provides a strategy suitable for high throughput analysis of cell function and especially gene function. One aspect of the present invention provides methods and arrays for creating transfection microarrays that are suitable for rapidly screening large sets of sample molecules for those causing or influencing cellular phenotypes of interest.

### BACKGROUND OF THE INVENTION

Conventional cell array screening methods either employ microtiter plates or take advantage of spotted carriers on glass surfaces containing the sample molecules such as siRNAs of interest. Basically, there are two traditional and common “cell array plate” technologies in use for cellular RNAi screening:

One method uses well-based, standard microtiter plates (such as 96- or 384-well microtiter plates). This method has the disadvantage that a high throughput of sample compounds is not feasible. These methods have a relative low capacity of transfection samples (maximal spot numbers limited to amount of wells/plate). These methods also need relative high amounts of material (e.g. siRNA and cells). Furthermore, uniformity is reduced, especially during large scale screenings, since individual wells need to be handled on many plates.

For example if a customer wants to perform a genome wide screening with 23000 genes and 4 siRNAs per gene, 82 siRNA per 96-well plate (controls on each plate) would result in 1122 96-well plates for one single experiment. This obviously has disadvantages regarding hands-on-time and costs. Also, storage room must be provided.

However, in the absence of suitable alternatives this format is the widely used format for RNAi screening. Due to the workload to handle over 1000 plates, RNAi screening is limited to a few laboratories with the appropriate size and equipment.

A different transfection method uses wall-less, standard arrays on (glass)-slides (see e.g. Ziauddin and Sabatini, 2001, Mousses et al., 2003, WO 02/0777264). This method, however, has also several disadvantages. The use of suspension cells is not possible and the possibility to perform on-spot processing (e.g. cell lysis for further biochemical investigations) is rather limited. Furthermore, the nucleic acid needs to be fixed on the spot to prevent lateral diffusion and-contamination during an experiment (e.g. by using a matrix or gelatine). The spotting of the sample nucleic acid for example, such as a siRNA, is performed with spotting equipment developed for DNA microarray for gene expression profiling study. There, DNA spotting has been proven not to be robust enough for reliable experiments. For instance the size of spots, the spot format and the purity of the glass slides varies, just to mention a few limiting factors. The market penetration of microarray based on DNA spotting has been reduced from approximately 50% to roughly 5% due to these problems.

### SUMMARY OF THE INVENTION

It is the object of the present invention to provide an improved transfection method and microarray transfection array system.

This object is solved by a transfection method of introducing sample molecules into cells, comprising at least the following steps:
(a) using a substrate comprising at least one sample spot area which is surrounded by an hydrophobic area;
(b) wherein sample molecules are applied to said at least one sample spot area, thereby placing said sample molecules in the discrete location of said sample spot area;
(c) applying cells to be transfected onto the substrate and incubation under appropriate conditions for entry of the sample molecules into said cells;
(d) whereby at least a portion of said sample molecules are introduced into said cells.

### BRIEF DESCRIPTION OF THE DRAWINGS

Details of the invention are outlined in the following drawings which are only presented as an example:

FIG. 1: Schematic cross section of the proposed microarray according to the invention according to a preferred embodiment.

FIG. 2: Scanning electron micrograph of a nano-structured ZrO₂ layer suitable for creating the hydrophobic and/or ultraphobic area(s). Theta is approximately 155° (water, SAM perfluorodecane triethoxysilane).

FIG. 3: Details of the surface chemistry according to one embodiment.

FIG. 4: Flowchart of the manufacturing process according to a preferred embodiment of the invention.

FIG. 5: Shows a schematic design of an array according to the present invention, wherein a sample droplet comprising sample molecules, such as siRNA, is applied. As can be seen, the sample droplets are confined by wettable spots on an ultraphobic surface.

FIG. 6: Schematic design of the dimensions of an array according to a preferred embodiment of the present invention.

FIG. 7: Schematic design of the dimensions of an array according to a further preferred embodiment of the present invention.

FIG. 8a: Shows the surface chemistry at the sample spots according to a preferred embodiment of the present invention as is also presented in FIGS. 6 and 7.

FIG. 8b: Shows the surface chemistry outside the sample spots according to a preferred embodiment of the present invention as is also presented in FIGS. 6 and 7.

### DETAILED DESCRIPTION OF A PREFERRED EMBODIMENT

Core of the present invention is the use of a microarray substrate for transfection, which comprises sample spot areas at least partially surrounded by hydrophobic areas. Preferably, the sample spot areas are completely encompassed and thus surrounded by the hydrophobic area. This can be achieved, for example, by placing a hydrophobic ring around the sample spot areas. However, preferably, basically the whole substrate surface is hydrophobic except for the sample spot areas, which are wettable and hence preferably hydrophilic and/or oleophilic.

This creates a well-less hydrophobic transfection array wherein the sample spot areas are securely isolated from each other due to the presence of the hydrophobic area(s) that surround(s) the sample spot areas. When compared to standard transfection arrays, the transfection array according to the present inventions has a considerably reduced risk of cross-contaminations due to hydrophobic area(s) around the sample spot areas. These features prevent liquid solutions and cell culture media from diffusing to neighbouring areas, thereby avoiding cross-contaminations. Each sample spot area is contacted by its own liquid compartment, which is isolated from the other sample spot areas by the surrounding hydrophobic area(s). The transfection array substrate, carrying the sample molecules placed on the sample spot areas can advantageously be provided by a supplier and can thus be pre-manufactured if desired. The transfection arrays may thus by provided as a ready to use product to the customer who basically only needs to apply the cells to be transfected.

The transfection cells that are being placed and usually grow on the sample spots take up the sample molecules, thereby creating spots of localized transfection (transfected cells) on the sample spot areas. However, with the transfection array according to the present invention, the cells do not create a cell lawn on the entire substrate surface as known with transfection arrays of the prior art. Instead, the cells are predominantly focussed within the boundaries of the hydrophobic area(s) on the sample spot areas.

Besides the advantages regarding the avoidance of cross-contaminations between sample spot areas, the advantage of the array transfection system according to the present invention is that the spot numbers can be considerably increased due to discrete and separated sample spot areas. These properties enable the construction of an array consisting of a very large number of samples. According to one embodiment, the substrate comprises at least 10, at least 50, at least 100, at least 250, at least 500, at least 1,000, at least 5,000, at least 7,500, at least 10,000, at least 15,000, or at least 20,000 sample spot areas. Preferably, the hydrophilic and/or oleophilic areas are arranged on the substrate according to a specific pattern. Thus, for example, a raster, a so-called array, can be produced in which the hydrophilic and/or oleophilic sample spot areas can then be easily moved forward for series tests, by, for example, a machine. The transfection arrays according to the present invention are thus automatically readable. It is preferred to have a minimum of 384 spots per plate in micro-titer plate format. Depending on the size of the array, spot numbers up to several thousands (25,000 and even more) are generally possible. For the desired 25,000 (or more) spots per micro-titer plate (area of approximately 120×80 mm²) one yields e.g. a sample pitch of 620 μm allowing a spot diameter of approximately 300 μm.

The transfection array method according to the present invention is thus useful for screening and especially HTS (high throughput screening) of many different sample molecules as an automated processing.

Even for a much smaller number of sample spots, the transfection array design according to the present invention enables a clear spatial definition of the sample location where the preparation processes can take place. This function typically requires an extreme liquid repellent property unlike conventional repellent surfaces such as Teflon coatings. It is thus preferred according to the present invention that the hydrophobic area is also oleophobic. A surface having hydrophobic and oleophobic properties is neither wettable by water nor by oily liquids.

A surface having hydrophobic and oleophobic properties is named “ultraphob” or ultraphobic according to the present invention.

A further advantage of the system according to the present invention compared to well-based plates is also the reduced need to employ pipetting steps for experimental handling. This as the sample molecules can be provided to the customer pre-fixed or pre-applied to the sample spot areas of the array substrate. The customer only needs to apply the cells to be transfected, wherein the cells will automatically be focused on the sample spot areas due to the repellent nature of the hydrophobic and/or ultraphobic area(s).

The transfection method according to the present invention also opens up the possibility to screen suspension cells. The strict separation of the sample spots also allows the use of different experimental conditions on the same platform, when required (e.g. different transfection reagents, cell types, sample molecule such as siRNA concentrations, transfection agents, cell culture medium and the like). The variability is thus considerably increased.

Preferably, said sample spot areas are the only locations on the array that can be wetted. Therefore, said sample spot area has hydrophilic and/or oleophilic properties. Said hydrophilic and/or oleophilic areas are preferably areas on which a drop of water or oil can be deposited; i.e. a drop of water or oil, which is brought into contact with the hydrophilic and/or oleophilic area by e.g. a pipette system, remains there and detaches itself or can be easily detached from the pipette system. Preferably, a drop of water or oil with a volume of 10 μl on the hydrophilic and or oleophilic areas have a contact angle <120° preferably <110° especially preferred <90° and/or the receding angle of this drop exceeds 10°.

The areas surrounding the samples spots are extremely repellent to liquids that are applied to the array during sample preparation. These areas are preferably ultraphob. Thus, the entire array operates as a well-less microarray where the sample compartments are defined by wettable areas in an otherwise non-wetting environment.

It has been also shown that the distribution of the sample material that is deposited on the wettable sample spot areas surrounded by the ultraphobic non-wetting environment is much more homogeneous compared to conventional hydrophobic surfaces. In the latter case, the inhomogenity of deposited material of a dried sample spot is due to largely different evaporation conditions between the rim and the center of the spot when the solvent evaporates, yielding more material deposited at the rim. Therefore, ultraphob surrounding areas are preferred in the context of the present invention.

Furthermore, the extreme repellency of the area surrounding the sample spots minimizes non-specific binding of samples on areas outside the spot, even if the liquid sample temporarily gets in contact with those areas. Hence, there is only a minimum of non-specific binding of sample molecules and constituents of cell culture media on areas outside the sample spot areas. Ultraphobic surfaces with 3-phase interfaces consist of entrapped air at the liquid-solid interface thereby reducing the true liquid-solid contact area to a fraction of a percent of the geometrical contact area. For example, at a contact angle of 178° the solid-liquid fraction is only 0.1% of the geometrical contact area.

According to one embodiment, said hydrophobic or ultraphobic area depicts a contact angle in relation to water of at least 140°, preferably at least 150°. An ultraphobic surface for the purpose of the invention preferably has a contact angle of a drop of water and/or oil that is on the surface more than 150°, preferably more than 160°, and especially preferred more than 170°. Preferably, the receding angle does not exceed 10°. Receding angle means the angle of inclination of a basically planar but structured surface from the horizontal, at which a stationary drop of water and/or oil with a volume of 10 μl is moved due to gravity for an inclination of the surface. Such ultraphobic surfaces are revealed, for example, in WO 98/23549, WO 96/04123, WO 96/21523, WO 99/10323, WO 00/39368, WO 00/39239, WO 00/39051, WO 00/38845 and WO 96/34697 which are hereby incorporated as references and thus count as part of the disclosure of the present invention.

Said hydrophobic and/or ultraphobic areas that can be used for creating the transfection arrays according to the present invention depict preferably a nano-structured topography. Preferably, said hydrophobic or ultraphobic area has a surface topography, where the topological frequency f of the individual Fourier components and their amplitudes a(f) expressed by the integral S(log (f))=a(f)·f calculated between the integration limits log (f₁/μm⁻¹)=−3 and log (f₂/μm⁻¹)=3 is at least 0.3 and which is made of an hydrophobic or particularly oleophobic material or is coated with a durable hydrophobic and/or particularly durable oleophobic material. Such an ultraphobic surface is described in the international patent registration WO 00/39249, which is hereby incorporated as a reference and thus counts as part of the disclosure.

The hydrophilic and/or oleophilic sample spot areas may be produced on the hydrophobic or ultraphobic surface, for example, through chemical and/or mechanical removal of at least a portion of the layer thickness of said repellent layer, preferably by means of a laser. They may also be deposited onto the hydrophobic or ultraphobic layer. Preferably, the hydrophilic and/or olephilic areas are, however, formed by way of modification of only the uppermost molecular layer of the hydrophobic or ultraphobic surface. Preferably, this modification is a mechanical and/or thermal ablation, by which preferably maximally one molecular layer of the hydrophobic or ultraphobic is removed. Furthermore, the modification preferably proceeds through the thermal or chemical change of the ultraphobic surface, however, without removal thereof, such as, for instance, as is described in DE 199 10 809, herein incorporated by reference. With this modification, the ultraphobic surface remains significantly unchanged in terms of its layer thickness. In a further preferred embodiment, the hydrophilic and/or oleophilic areas are reversibly producible on portions of the ultrahydrophobic surface.

Preferred ultraphobic materials that can be used to create the ultraphobic material are, for example, nanostructured ZrO₂ or Al₂O₃ layers. They can be applied to the substrate by sputter deposition. As surface chemistry on top of the nanostructured layer, SAM molecules can be used. Suitable molecules are, for example, fluorinated silanes, fluorinated phosphates and phosphonates, fluorinated iodides and fluorinated fatty acids. Preferably, the surface chemistry is at least a binary mixed monolayer of chains, having a different length (mixed on a molecular scale). According to one embodiment, the surface comprises a mixed monolyer of C10:C12 fluorinated alkyl chains. According to a further embodiment, a mixed monolyer of C10:C20 fluorinated alkyl chains is used. Fluorinated alkyl chains comprise a helical conformation and are rigid. An advantage of fluorinated monolayers is, that they do not phase separate.

The sample spot area preferably comprises or consists of SAMs, which can be single component or multicomponent. They may also be coated with peptides, such as fibronectin. The hydrophilic surface chemistry comprises preferably one compound selected from the group consisting of 3-aminopropyl-triethoxysilane, 3 mercaptopropyl-trimethoxysilane, Methacryloxypropyl-triethoxysilane, Hexadecyltrimethoxysilane, 2-[Methoxy(polyethylenoxy)propyl]trimethoxysilane, 1 H, 1 H, 2H, 2H Perfluordecyltriethoxy-silane and 3-(phenylamino)propyltrimethyldiethoxysilane. Preferably, the compound is selected from 3-aminopropyl-triethoxysilane, 3 mercaptopropyl-trimethoxysilane and 3-(phenylamino)propyltrimethyldiethoxysilane as these compounds proved to be suitable to allow cell growth of a large variety of cells such as HeLaS3, MCF-7, HEK293, HUVEC, CaCO-2 and HepG2. The cells adhered well.

Examples of sample molecules that may be applied onto the sample spot areas for the transfection reaction include but are not limited to:
nucleic acids, thereunder DNA, RNA and DNA/RNA hybrids, wherein the nucleic acids may be single-or doublestranded as well as linear or circular;
peptides
proteins
sugars
lipids
polysaccharides
organic molecules, especially small molecules.

The sample molecules are applied to the array by any suitable methods, such as for example, spotting with pin-tools, dispensing technologies such as dispensing well plates or inkjet technologies.

RNA molecules and in particular RNAi mediating compounds such as siRNA or shRNA molecules are especially preferred sample molecules for the transfection array of the present invention. By applying a library of RNAi mediating compounds such as siRNAs to the transfection array according to the present invention, screening and hence analysis of the effects mediated by said siRNAs is considerably improved. It is even possible to provide a genome library onto the array of the present invention.

It is known that siRNA molecules (double-stranded (i.e. duplex) short interfering RNA having a length of 21-30 nucleotides (nt) with terminal 3′-overhangs of 2 nucleotides) can be used to post-transcriptionally silence gene expression in mammalian cells (Elbashir et at, Nature 2001, 411:494-498). Complexing of siRNAs with suitable transfection reagents and application of these complexes onto the cells results in endocytotic uptake of the siRNA- complexes. SiRNA is finally released into the cytoplasm and the siRNA molecules are recognized and incorporated into a complex called RISC (RNA induced silencing complex). A RISC associated ATP-depended helicase activity unwinds the duplex and enables either of the two strands to independently guide target mRNA recognition (Tuschl, Mol. Interv. 2002, 2:158-167).

The RISC complex then recognizes a region within the mRNA to which the siRNA sequence is complementary and binds to this region in the mRNA. The mRNA is then endonucleolitically cleaved at that position, where the RISC complex is bound, in a final step, the endonucleolitically cleaved mRNA is degraded by exonucleases. This mechanism of siRNA mediated gene silencing (RNA interference, RNAi) is widely used to perform knockdown experiments in eukaryotic cell cultures.

It is also known that not every siRNA duplex is able to knockdown the mRNA level to the same degree. Some siRNAs are able to knockdown the initial level to approx. 10-20%, some others to intermediate levels (20-70%), and still others are not able to knockdown the mRNA level to a measurable level at all. There are certain design rules (so called “Tuschl-rules”) for the design of siRNAs, but even siRNAs designed according to these rules show the above described variability. The reason for this observation is not known. It is believed that also the thermodynamic stability of the duplex may play a role in the efficiency of the siRNA to induce silencing. This variability as well as the fact that even “good” siRNAs are not able to completely knockout all mRNAs is a severe disadvantage of the siRNA technology (Gong D., Trensd. Biotechnol. 2004, 22:451-454; Khvorova A, Cell 2003, 115:209-216; Schwarz D. S., Cell 2003, 115:199-208; Reynolds A., Nat. Biotechnol. 2004, 22:326-330).The search for more potent gene silencing either by enhancement of uptake into the cells or by more potent intracellular siRNA duplexes is therefore central to the field of siRNA research.

Moreover, it is known that several chemical modifications have been tested in order to increase the ability of siRNA molecules to induce silencing and also to increase their stability. Chemical modifications of siRNA molecules or oligonucleotides (ODN) are affecting the nuclease stability of these molecules or their affinity to target mRNAs (Manoharan M., Curr. Opin. in Chem. Biol. 2004, 8:570-579). Different types of modifications have been described to improve the stability against serum nucleases and/or to improve the affinity to targets. Described are: a. 2′-OH modifications with halogen, amine, —O-alkyl, —O-allyl, alkyl-groups; b. internucleotide linkages, such as orthoester, phosphate ester, phosphodiester, -triester, phosphorothioate, phosphorodithioate, phosphonate, phosphonothioate, phosphorothiotriester, phosphoramidate, phosphorothioamidate, phosphinate, and boronate linkage, ether-, allyl ether-, allyl sulfide-, formacetal/ketal-sulfide-, sulfoxide-, sulfone-, sulfamate-, sulfonamide-, siloxane-, amide-, cationic alkylpolyamine-, guanidyl-, morpholino-, hexose sugar or amide-containing linkage, or a two to four atom linkage; c. conjugates, such as aminoacids, peptides, polypeptides, protein, sugars, carbonhydrates, lipids, polymers, nucleotides, polynucleotides, as well as combinations thereof, passive delivery with cholesterol conjugates.

Normal (unprotected) siRNAs may show a low stability in cell culture media and in body fluids, such as serum. This results in a risk of degradation of siRNA during the transfection or the systemic delivery. Accordingly, due to the degradation, less siRNA remains in the cells for efficient silencing, and the degraded (shortened) siRNA may lead to off-target effects in cells due to unspecific base pairing. Transfection of siRNA to cells is shown to lead to off-target effects. Specifically, high amounts of siRNA may lead to PKR activation (PKR: Protein Kinase R; double stranded RNA-activated protein kinase). Also, the recognition and incorporation of the sense strand in the RISC instead of the antisense strand might lead also to off-target effects.

Therefore, it is preferred according to the present invention to use improved siRNA molecules as sample molecules that are protected against degradation so that the gene silencing activity of the siRNA molecules and hence the transfection efficiency is improved and the amount of siRNA molecules needed for efficient gene silencing and successful transfection is decreased.

The chemically modified siRNA molecules enhance the stability of these molecules in culture media. The degradation of siRNA is effectively suppressed, and the full length siRNA will be more active and lead to no off-target effects. Moreover, the sensitivity of the siRNA is enhanced. Lower amount of siRNA will be needed to achieve a high level of gene silencing in the transfection method according to the present invention. The siRNA may be incorporated into the RISC in the right orientation, so that only the antisense strand will be able to induce silencing, which leads to higher specificity. In addition, the sense strand is inactivated due to modifications, resulting in the elimination of off-target effects. The siRNA according to the present invention enables a more efficient unwinding of the siRNA duplex and hybridization with the target mRNA, leading to an improved target binding affinity. Furthermore, the transfection efficiency of the siRNAs is enhanced.

Chemically modified siRNA molecules with the combination of the following groups and linkages at the well defined positions of both the sense and antisense strands of the siRNA improve stability, sensitivity and specificity of the siRNA:
(a) 2′-deoxy modified nucleotides;
(b) 2′-methoxy modified nucleotides;
(c) two nucleosides linked by a 3′ to 5′ or 2′ to 5′ formacetal linkage;
(d) nucleotides modified at the 2′-position by a —O—CH₂—O—(CEb)₂—OH group; and
(e) nucleotides comprising in the 3′-[rho]osition a —O—CH₂—O—(CH₂)7—CH₃ group.

Further advantageous features of siRNAs that can be used as sample molecules in a transfection method according to the present invention are described in WO2006/102970, herein incorporated by reference.

The sample molecules to be transfected into the cells are either covalently or non-covalently bound/attached to the sample spot areas. The sample molecules, e.g. nucleic acids such as siRNA molecules can be synthesized prior to the application to the sample spot area. Application to the array surface is performed preferentially by simple spotting sample molecules containing solutions, optionally followed by a drying process. Alternatively, spotting can be supported by various surface chemistries and/or chemical carrier materials or matrices.

The sample molecules may thus be mixed with a suitable matrix or polymer in order to enhance binding/adherence of the sample molecules to the sample spot area. The matrix respectively polymer may also be pre-applied before the sample molecules are added. According to one embodiment, said sample molecules are embedded in a matrix at the sample spot area. These matrices may be synthetic or natural and can be chosen from gelatine, agar or agarose, silanes, polyD lysines, (poly)acrylamide, antibodies or fragments of thereof, synthetic (poly)peptides, lipids, crude or purified preparations of cellular proteins, sugars or polysaccharides, extracellular matrix components, such as collagen, fibronectin, matrigel, anorganic ions, other polymers in order to enhance binding of the sample molecules to the sample spot area.

Also other reagents, such as for example cytotoxicity reductive reagents, cell binding reagents, cell growing reagents, cell stimulating reagents or cell inhibiting reagents or the compounds/media for culturing specific cells, can be also affixed or applied to the sample spot area.

The sample spot areas may be designed such that the transfection cells adhere thereto in order to promote cell growth. According to one embodiment, the surface properties of at least one sample spot area are modified to enhance the adherence of the cells to said sample spot area. Hence, depending on the specific demands of the application/transfection assay the spot area may be modified to increase cell binding and proliferation. This can, for example, be achieved by a fraction of phosphonic acids exposed at the surface of the spot to allow for binding of the cell attractive peptide derivative RGDC using Zirconium alkoxyde complexes through (maleimido)-alkoxycarboxylate intermediates. Such surfaces modified with RGDC have been shown to be effective for osteoblast binding and proliferation (M. P. Danahy, M. J. Avaltroni, K. S. Midwood, J. E. Schwarzbauer, J. Schwartz; Self-assembled Monolayers of alpha-omega-Diphosphonic Acids on Ti Enable Complete or Spatially Controlled Surface Derivatization; Langmuir 20, 5333 (2004)).

According to an in situ synthesis protocol, which can be especially used with nucleic acids as sample molecules, the nucleic acids such as, siRNA, are directly synthesised on the surface of the slide, for example, by photolithographic methods or inkjet printing of nucleotides, for example. This avoids separate spotting steps.

In order to support the sample molecule uptake, several transfection methods can be used that facilitate the entry or uptake of the sample molecules such as the siRNAs/NA into the cells. Preferentially, this is achieved by chemical methods such as lipofection, dendrimers, or cell membrane penetrating peptides. However, it can also be performed by physical methods, such as electroporation, shot gun-, or laser supported transfection, or by biological methods, such as viral- or bacterial vectors or pore-forming toxins. The transfection reagent or delivery reagents are preferably cationic compounds that can introduce the sample molecules, such as nucleic acids, proteins, peptides, sugars, polysaccharides, organic compounds, and other molecules into cells. Preferred embodiments use cationic oligomers, such as low molecular weight polyethyleneimine (PEI), low molecular weight poly (L-lysine) (PLL), low molecular weight chitosan, or low molecular weight dendrimers. According to their modular composition, reagents can be classified as: lipids, polymers, lipid-polymers and/or their combinations and/or their derivatives, which contain a cell-targeting or an intracellular-targeting moiety and/or a membrane-destabilizing component, as well as delivery enhancers. Also electroporation, membrane penetrating peptides and nano particles can be used.

Preferably, the surface chemistry of spots and of areas surrounding the spots are stable at pH 7.5+/−1. Furthermore, it is preferred that the entire chip/array is stable in aqueous cell culture media for 5 days at 37° C. Furthermore, the materials in contact with the samples are preferably not cytotoxic.

In particular, sample spots are compatible to the application of aqueous transfection reagents containing at least one of the following compounds: Lipids, such as DOPE (dioleoyl phosphatidyl ethanolamine); DOTMA (1,2-dioleyloxypropyl-3-trimethyl ammonium bromide); DMRIE (1,2-dimyristyloxypropyl-3-dimethyl-hydroxyethyl ammonium bromide); DDAB (dimethyldioctyldeyl ammonium bromide); DOTAP (1,2-dioleoyloxy-3-(trimethylamino)propane); DC-CHOL ((3beta[N″,N″-dimethylaminoethane)carbamoy1]-cholesterol); DOGS (5-carboxyspermylglycine dioctadecylamide); DPPES (Dipalmitoylphophatidylethanolamine-6-carboxyspermylamide); Pyridinium amphiphiles such as N-Methyl-4-(dioleyl)methylpyridinium chloride. Preferred agents are RNAifect® and HiPerfect® distributed by Qiagen.

According to one embodiment, the transfection reagent is applied to the substrate either before the cells are applied to the substrate or together with the cells, in form of a transfection reagent/cell mixture. This embodiment has the advantage that the user may use the transfection agent of his choice that works best for the cells to be transfected. According to a different embodiment, the transfection reagent is applied together with the sample molecules onto the sample spot areas and is thus also pre-fixed respectively pre-applied to the sample spot areas. This embodiment has the advantage that the user only needs to apply the cells onto the array. Furthermore, stabilisation of the sample molecules can be achieved by this embodiment.

According to a very preferred embodiment, at least said sample spot area is optically transparent or translucent. However, the whole substrate may also be optically transparent or translucent. This feature has the special advantage that the sample spot areas are designed suitable for optical analysis method such as in transmission light microscopy. Therefore, at least one sample spot area, or at least two or even substantially all sample spot areas are optically transparent or translucent and provide optical properties similar to conventional microscopy slides in order to allow the analysis of the results by optical microscopes and fluorescent readers. In this respect it is also advantageous that the array formats are designed compatible to instruments such as transmission optical microscopes and fluorescent readers. This also allows an automated process.

It is apparent that each sample spot may carry a different sample molecule in order to be able to perform the described screening processes. For example, each sample spot area of the transfection array substrate according to the present invention may carry a different siRNA in order to analyse the influence of the different siRNAs on the cells. According to a further embodiment, at least two different kinds of sample molecules are used for one transfection reaction. This embodiment has the advantage that a combinatoric transfection assay is feasible. According to one embodiment, the different kinds of sample molecules are all applied to and are thus pre-fixed respectively pre-deposited to the sample spot areas. For example, a nucleic acid such as a siRNA may be applied on the sample spot area together with a different sample molecule such as a different siRNA or a pharmacological agent, such as a small molecule. Two different sample molecules are thus prefixed respectively pre-deposited on one sample spot area. The later embodiment using a pharmacological agent such as a small molecule, for example, is especially advantageous in case the applied siRNAs do not provide a complete silencing effect on the gene expression of interest. By combining the siRNA with another sample molecule that influences the same pathway, an improved or complete interference and hence silencing effect may be achieved (see e.g. Morgan-Lappe et al, Oncogene (2006) 25, 1340-1348; Giuliano et al, Journal of Biomolecular Screening 9(7); 2004). Also cross-interactions of different sample molecules may be thereby analysed. This embodiment also allows using lower concentrations of said siRNAs (as an example of a first sample molecule) and said second sample molecule. This feature is especially advantageous in case an inhibitory effect of one of the compounds is only achieved at toxic concentrations of said compound.

Alternatively to applying said second molecule in advance to the sample spot areas, said second sample molecules may also be applied prior to or after applying the cells to the substrate or may be pre-mixed with the cells. This embodiment has the advantage that the user is free to choose appropriate second sample molecules.

According to a further embodiment, the influence of the sample molecules on the cell activity may be observed by using the transfection method according to the present invention. In one embodiment, the transfection screen can be used to analyse the inability to grow or survive when a parasite or infectious agent such as a virus is added to the cell of interest. In this case the selection would be for knock-outs that are targeting genes that are specifically essential for some aspect of viral or parasitic function within a cell that are only essential when that cell is infected. Since some viral infections result in the induction of survival factors (such as CrmA, p35) it is likely that at least some cell functions are different and potentially selectively needed during viral, parasite growth. It is thus possible to study viral cycles in further detail. According to one embodiment, a transfection substrate is used, comprising the sample molecules such as siRNA on the sample spot areas. The cells are then placed onto the transfection substrate and incubated such that transfection occurs. After the sample molecules are transfected in the cells long enough to confer an effect, viral agents are applied to the cells. This setting allows studying, for example, the viral cycle and the influence of certain messengers on the viral reproduction cycle in more detail.

The cells to be transfected according to the teachings of the present invention may be of any nature. Examples are eukaryotic cells, such as mammalian cells (e. g., human, monkey, canine, feline, bovine, or murine cells), bacterial, insect or plant cells.

The eukaryotic cells are preferably mammalian cells. The mammalian cells may be dividing cells or non-dividing cells and the cells may be transformed cells or primary cells. The mammalian cells may be somatic cells or stem cells. Detecting cells into which the sample molecule has been delivered may be performed by detecting the sample molecule itself, its product, its target molecule, the products catalyzed or products regulated by the sample molecule or the effects on the phenotype of the cells. The cells are plated (placed) onto the surface bearing the sample molecules in sufficient density and under appropriate conditions for introduction/entry of the sample molecules into the cells and to allow interaction with the cellular components necessary for conferring an effect.

The cells are preferably applied when the sample molecules are already placed on the sample spot areas. Any application mode can be used, such as for example, parallel transfer of the liquids with a carriage, stamping technologies and spotting technologies.

Detection of effects on recipient cells (cells containing said sample molecules introduced by transfection) can be carried out by a variety of known techniques, such as immunofluorescence, in which a fluorescently labelled antibody that binds a protein of interest (for example, a protein thought to be encoded by a transfected DNA or a protein whose expression or function is altered through the action of the introduced sample molecule) is used to determine the effects on the cells. A variety of methods can be used to detect the consequence of sample molecule uptake, and in many embodiments, expression (at least transcription) of the introduced molecules or the effects mediated by the sample molecule (for example, interference in case of siRNA). In a general sense, the assay provides the means for determining if the sample molecule is able to confer a change in the phenotype of the cell relative to the same cell lacking the sample molecule. Such changes can be detected on a gross cellular level, such as by changes in cell morphology (membrane ruffling, rate of mitosis, rate of cell death, mechanism of cell death, dye uptake, and the like). In other embodiments, the changes to the cell's phenotype, if any, are detected by more focused means, such as the detection of the level of a particular protein (such as a selectable or detectable marker), or level of mRNA or second messenger, to name but a few. Changes in the cell's phenotype can also be determined by assaying reporter genes (beta-galactosidase, green fluorescent protein, beta-lactamase, luciferase, chloramphenicol acetyl transferase), assaying enzymes, using immunoassays, staining with dyes (for example, DAPI, calcofluor), assaying electrical changes, characterizing changes in cell shape, examining changes in protein conformation, and counting cell number. Other changes of interest could be detected by methods such as chemical assays, light microscopy, scanning electron microscopy, transmission electron microscopy, atomic force microscopy, confocal microscopy, image reconstruction microscopy, scanners, autoradiography, light scattering, light absorbance, NMR, PET, patch clamping, calorimetry, mass spectrometry, surface plasmon resonance, time resolved fluorescence. Data could be collected at single or multiple time points and analyzed by the appropriate software.

Hence, the results of the sample molecule delivery can be analyzed by different methods. Furthermore, the transfected cells can be directly processed further, especially if a standard array format (see above) is used. For example, if the delivered sample molecules can modulate gene expression, the target gene expression level can also be determined by methods such as autoradiography, in situ hybridization, and in situ PCR. The identification/processing method depends on the properties of the delivered sample molecules, their expression product, the target modulated by it, and/or the final product resulting from delivery of the sample molecules.

Any suitable surface that can be used to affix or adhere the sample molecules to its surface can be used for the purpose of the present invention. Binding of the sample molecules may be covalent or non-covalent. For example, the surface can be glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose), biomaterials and minerals (such as hydroxylapatite, graphite). According to preferred embodiments, the surfaces are slides (glass or poly-L lysine coated slides). As outlined above, it is especially preferred that a transparent or translucent material is used, wherein at least one, respectively all sample spots are optically transparent or translucent. Suitable transparent or translucent materials are, for example, borosilicate or translucent metals.

According to one embodiment, a transparent or translucent substrate with a reduced light-scattering, ultraphobic surface is used that has a total scatter loss of ≦7%, preferably 3%, particularly preferably ≦1%. Said substrate preferably has a contact angle in relation to water of at least 140° preferably at least 150° and a roll-off angle of ≦20° , preferably ≦10. Respective reduced light-scattering surfaces are e.g. described in U.S. patent application Ser. No. 2006/0159934, especially in paragraphs 24 to 26, herein incorporated by reference. This document also describes suitable coatings for obtaining respectively nanostructered surfaces, suitable for creating an ultraphobic surface. For example, a coating made of oxides, fluorides, carbides, nitrides, selenides in particular of metals such as zirconium, titanium, aluminium and tantalum. Oxides such as a zirconium oxide layer are especially preferred. Details regarding the materials and possible processes for applying said layers are also described in U.S. patent application Ser. No. 2006/0159934, paragraphs 89 to 150, herein incorporated by reference.

In order to improve the hydrophobic and/or ultraphobic properties of said area(s) surrounding the sample spot areas, it is preferred to provide the substrate with an additional coating of a hydrophobic or oleophobic phobing agent.

Hydrophobic or oleophobic phobing agents are usually surface-active compounds of any molar mass. These compounds are preferably cationic, anionic, amphoteric or non-ionic surface-active compounds, such as those listed, for example, in the dictionary “Surfactants Europa, A Dictionary of Surface Active Agents available in Europe, Edited by Gordon L. Hollis, Royal Society of Chemistry, Cambridge, 1995.

Examples of anionic phobing agents include: alkyl sulphates, ether sulphates, ether carboxylates, phosphate esters, sulphosuccinates, sulphosuccinate amides, paraffin sulphonates, olefin sulphonates, sarcosinates, isothionates, taurates and lignin compounds.

Examples of cationic phobing agents include: quaternary alkyl ammonium compounds and imidazoles.

Examples of amphoteric phobic agents are betaines, glycinates, propionates and imidazoles.

Non-ionic phobing agents are, for example: alkoxyates, alkyloamides, esters, amine oxides and alkylpolyglycosides. Also possible are: conversion products of alkylene oxides with compounds suitable for alkylation, such as for example fatty alcohols, fatty amines, fatty acids, phenols, alkyl phenols, arylalkyl phenols such as styrene phenol condensates, carboxylic acid amides and resin acids.

Particularly preferred are phobing agents in which 1 to 100%, particularly preferably 60 to 95%, of the hydrogen atoms are substituted by fluorine atoms, for example, are perfluorinated alkyl sulphate, perfluorinated alkyl sulphonates, perfluorinated alkyl phosphates, perfluorinated alkyl phosphinates, perfluorinated alkoxysilanes, perfluorinated chlorosilanes, perfluorinated alkoxychlorosilanes, perfluorinated thiols and perfluorinated carboxylic acids.

Further details of suitable phobing agents are disclosed in U.S. patent application Ser. No. 2006/0159934, paragraphs 108 to 112, herein incorporated by reference.

Also provided by the present invention is a transfection array substrate for introducing sample molecules into cells, comprising a substrate comprising several sample spot areas arranged in an array format wherein said sample spot areas are surrounded by a hydrophobic area and wherein said sample spot areas carry sample molecules, whereby said sample molecules are located in the discrete location of said sample spot areas.

Preferred properties and uses of a respective transfection array are discussed above in detail in conjunction with the transfection method described above. Further suitable surface properties for the different areas of the transfection array substrate were described in detail above. The above discussion which also applies to the transfection array. As outlined above, siRNA are preferably used as sample molecules that are applied to the array substrate. In order to allow screening methods, preferably at least parts of said sample spot areas carry different kind of sample molecules. In order to allow an optical analysis, a transparent or translucent substrate is preferred. However, for some embodiments it may be sufficient to render the sample spot areas transparent or translucent.

In order to allow easy handling by the user, it is preferred that the microarray and its functional specifications are stable up to 100° C., is sterilizable and freezable (at least −20° C.).

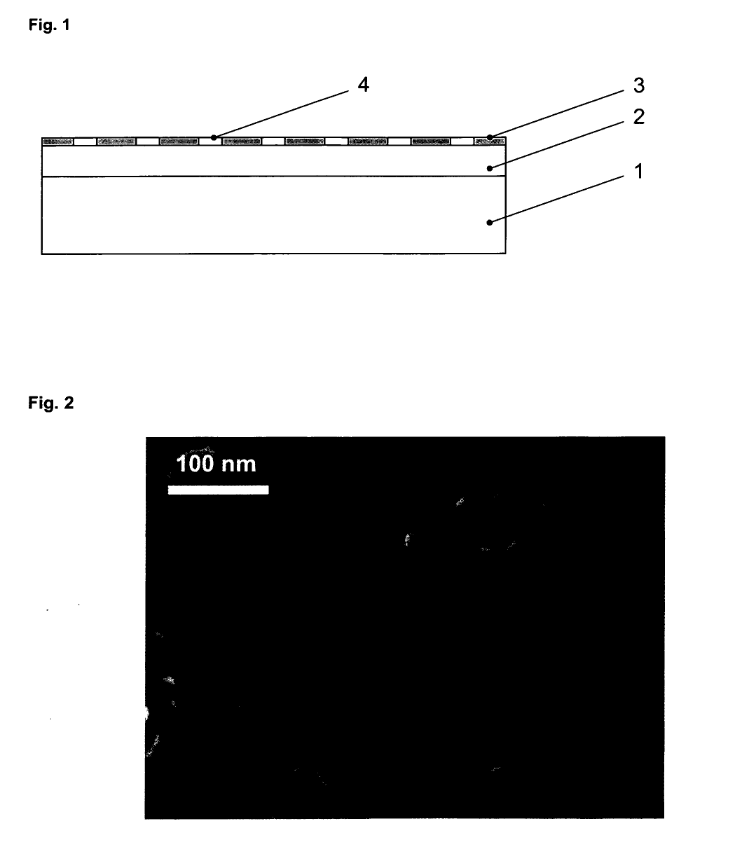

The different layers of the micro array depicted in FIG. 1 are represented by the following numbers:
(1) Substrate, preferably glass
(2) n-ZrO₂ layer, approx. 100 nm
(3) Self-assembled monolayer of a cleavable fluorinated compound, preferably approx. 1 nm
(4) Spots of immobilized sample molecules, preferably RNA (siRNA)

The substrates (1) according to the embodiment of FIG. 1 are conventional float glass substrates used and processed as individual parts in their final dimensions. Material selection is usually determined depending on the desired thickness and optical properties of the substrates desired, such as background fluorescence. All different glass materials can be used for subsequent processing in the same manner. A glass material due to the optic properties and especially its transparency, is a preferred substrate material according to the present invention as it allows an optical inspection of the transfection results.

On top of the substrate (1), a nano-structured zirconium oxide layer (2) is applied. The n-ZrO₂ layer in this example was deposited by reactive electron beam evaporation on a borofloat glass substrate (1) at 590 K. A similar surface topography is obtained by DC sputter deposition at 300 K substrate temperature yielding water contact angles of up to 155°. Said layer (2) delivers a topography that reveals ultraphobicity when chemically oleophobized by a suitable monolayer (3).

The n-ZrO₂ layer (2) as deposited by reactive electron beam evaporation or reactive DC sputter deposition delivers a static water contact angle of up to 155°. ZrO₂ is regarded as a preferred material for the nano-structured layer especially due to its stability in either acidic and basic environments during prolonged exposition. In contrast, even though suitable for certain embodiments, sputter deposited Al₂O₃ in optical quality has been tested and is not sufficient for fluorescent DNA arrays due to its amphoteric property.

As a second layer, a self-assembled fluorinated monolayer (3) is applied onto the nano-structured layer (2). It has two functions. It provides the surface chemistry with densely packed fluorinated chains to yield an ultra-hydrophobic surface property in combination with the n-ZrO₂ surface topography. In addition, the fluorinated chains of the monolayer are UV-cleavable so that the surface chemistry can be altered in a defined manner, both spatially and chemically, to yield the sample spot areas with their necessary or desirable functions (e.g. binding of the sample molecules, adherence of the transfection cells).

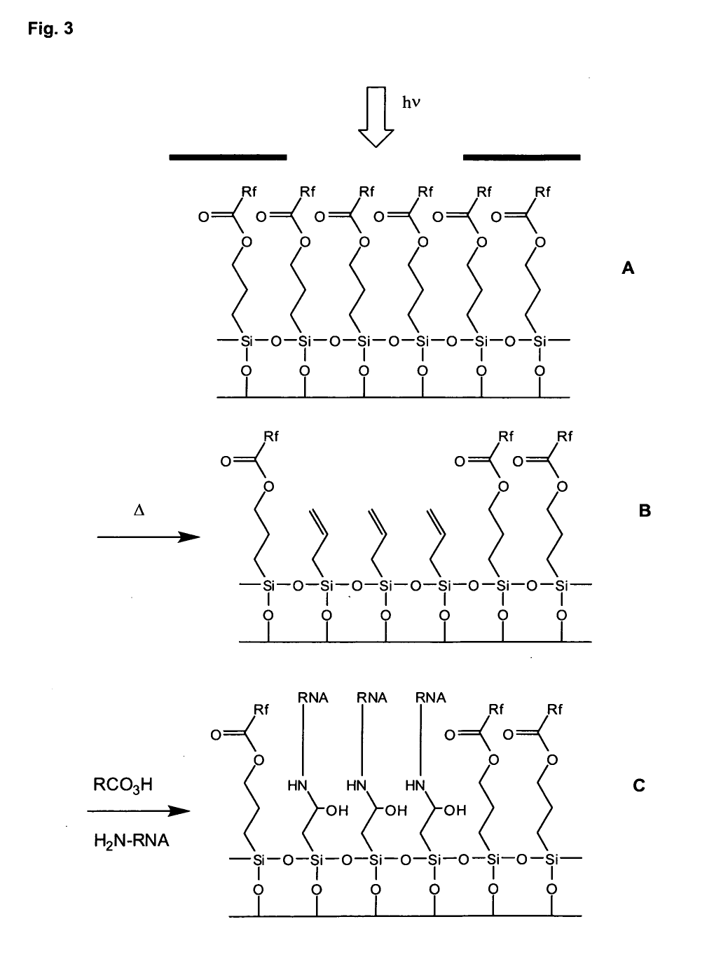

The structure of the monolayer of non-spot areas (A) is shown in FIG. 3. Here, a silane such as a substituted trichlorosilane has been adsorbed yielding a polysiloxane monolayer. These chemisorbed moieties are according to the shown embodiment present on both, sample spot and non-spot areas.

High-quality siloxane monolayers on oxide surfaces are generally regarded as difficult to produce in a reproducible manner. It is suggested that this is mainly due to the poor control of miniscule amounts of water in the adsorption solution (B. M. Silverman, K. A. Wieghaus, J. Schwartz; Comparative Properties of Siloxane vs Phosphonate Monolayers on a Key Titanium Alloy; Langmuir 21, 225 (2005)). Here, major improvements of process stability have been obtained by co-evaporation of trichlorosilanes and dimethylchlorosilanes in the presence of a defined water pressure using silicon dioxide as a substrate (A. Ulman; Formation and Structure of Self-Assembled Monolayers; Chem. Rev. 96, 1533 (1996)).

It may be, however, advantageous to replace the organosilicon derivatives by phosphonates because of their superior hydrolytic stability (H. Schift, S. Saxer, A. Park, C. Padeste, U. Pieles, J. Gobrecht; Controlled co-evaporation of silanes for nanoimprint stamps; Nanotechnology 16, 171 (2005)), and likely better process stability.

Dewetting properties of the hydrophobic or ultraphobic areas can be further optimized by molecular roughness obtained from self-assembled fluorinated monolayers (3). Randomly mixed perfluorinated carbon chains of a height difference of only 1.8 A, for example, decrease the repellency for liquids with low surface tension on surfaces significantly if they exhibit a surface that is already extremly rough. Such “additional” roughness with very high spatial frequencies can be assembled on top of a nano-scale topography thereby increasing its dewetting properties.

Such optimization can be performed using the perfluorinated alkyl chains (denoted as Rf in A, FIG. 3). Here, different long chains may significantly increase dewetting when mixed on the molecular scale. They can be introduced by using different long perlfuorinated acid chlorides. The necessary intermediates are commercially available in a variaty of chain lengths (C4-C18).

Sample spot areas (4) may be formed as depicted by a controlled chemical modification of the fluorinated monolayer initiated by UV light. A variety of such compounds using different chemical structures for photolithographic micropatterning are known, see for example (J. D. Jeyaprakash, S. Samuel, J. Rühe; A Facile Photochemical Surface Modification Technique fort he Generation of Microstructured Fluorinated Surfaces; Langmuir 20, 10080 (2004); K. Lee, F. Pan, G. T. Caroll, N, J. Turro, J. T. Koberstein; Photolithographic Technique for Direct Photochemical Modification and Chemical Micropatterning of Surfaces; Langmuir 20, 1812 (2004)). In the depicted example, a perfluorinated ester is proposed for this purpose. It has been shown that such ester moieties that are present as perfluorinated side chains in isoprene-styrene block copolymers can be thermally cleaved selectively at approx. 340° C. to yield the corresponding olefin by a thermally allowed pericyclic retro-en reaction (A. Böker, K. Reihs, J. Wang, R. Stadler, C. Ober; Selectively thermally cleavable fluorinated side chain block copolymers: surface chemistry and surface properties; Macromolecules 33, 1310 (2000)). Other structures such as carbonate and allophanate moieties are cleavable at similar temperatures (A. Böker, T. Herweg, K. Reihs; Selective alteration of polymer surfaces by thermal cleavage of fluorinated side chains; Macromolecules 35, 4929 (2002)). This thermal process can likewise be initiated by UV-irradiation.

An olefin (B) is formed (see FIG. 3) as a result of the selective decomposition that can be used for a defined chemical functionalisation of the sample spot area.

The siloxane monolayer is likely to be stable under such conditions, as alkylsiloxane self assembled monolayers consisting of CH₃(CH₂)ₙ chains are stable in vacuum to about 740 K independent of chain length (n=4, 8, 18) (G. J. Kluth, M. M. Sung, R. Maboudian; Thermal behavior of alkylsiloxane self-assembled monolayers on the oxidized Si(100) surface; Langmuir 13, 3775 (1997)).

The olefin (B) (see FIG. 3) can be epoxidized to yield a surface ready for immobilization of RNA (C), either covalently, but also non-covalently, for example, by drying sample molecules, such as siRNA containing solutions on the spot or, alternatively, by embedding the sample molecule within polymers or matrix compounds. A large variety of protocols exists for these processes for fixing sample molecules on a surface.

Depending on the specific demands of the application, the sample spot area may be modified to increase cell binding and proliferation. This can, for example, be achieved by a fraction of phosphonic acids exposed at the surface of the spot to allow for binding of the cell attractive peptide derivative RGDC using Zr alkoxyde complexes through (maleimido)-alkoxycarboxylate intermediates. Such surfaces modified with

RGDC have been shown to be effective for osteoblast binding and proliferation (see above).

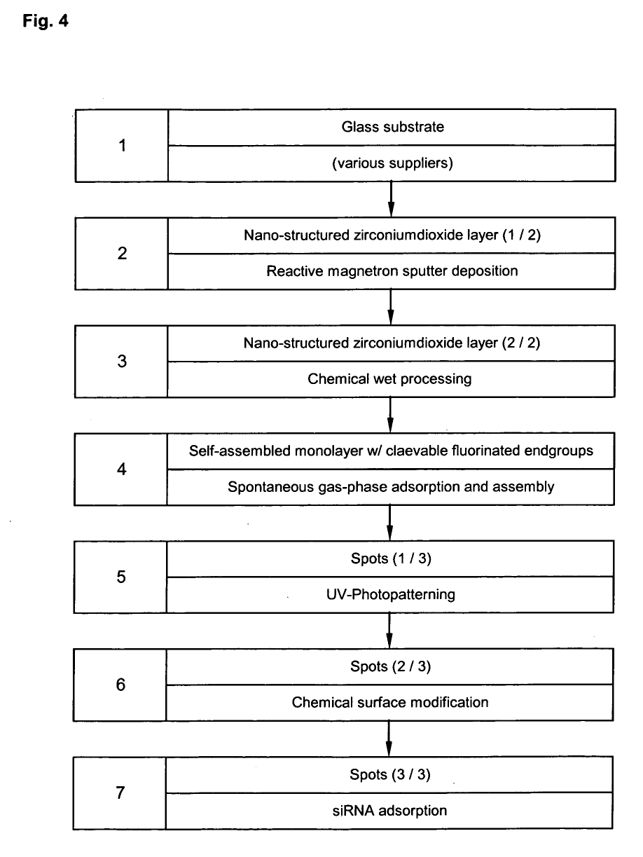

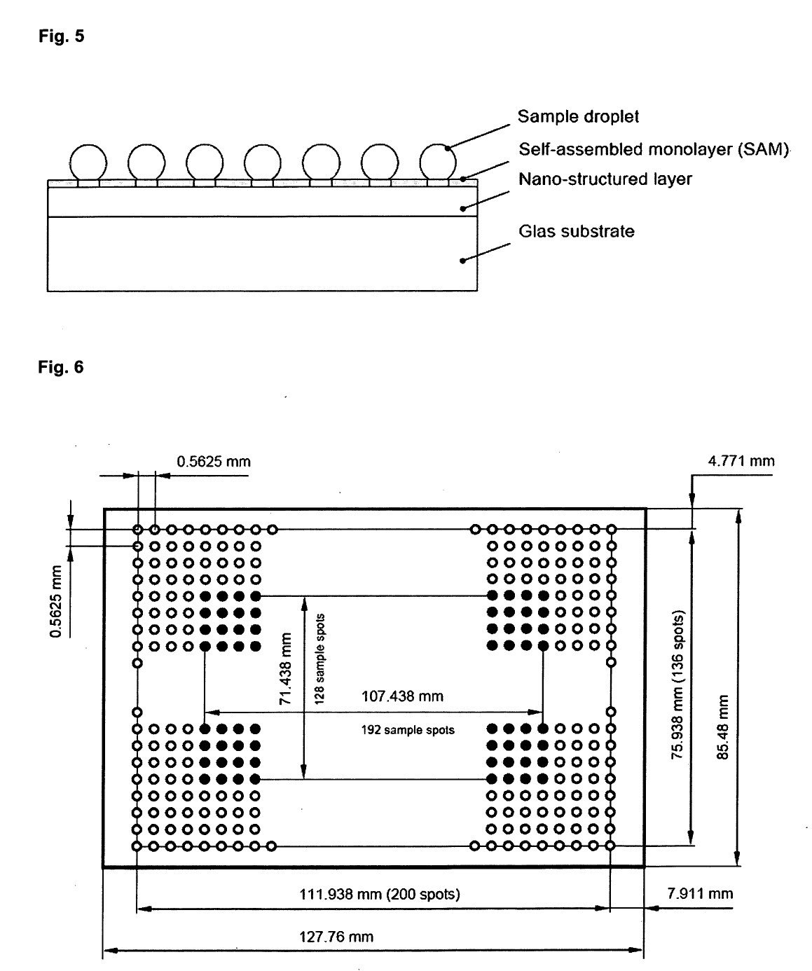

An overview of a manufacturing process as outlined for the microarray design is given in FIG. 4.

FIG. 6. shows a schematic drawing of the dimensions of an array according to a preferred embodiment. The array footprint is 127.76 mm×85.48 mm. The thickness of the array is approx. 1.1 mm (standard) but 0.145 mm when used for immersion objective lenses requiring a close distance. The total number of spots is 200×136=27,200 spots. The outermost spots of each row and column are preferably not used for samples. The number of sample spots is 192×128=24,576 spots. The spot diameter is 0.300 mm; the spot to spot distance (center to center) is 0.5625 mm. The density of the spots is 27,200 spots on 112.238 mm×76.238 mm=318 spots/cm². The sample volumes depend on the technique of liquid application. Usually, the volumes used are less than 7 nL (hemispheres). Typical volumes are about 4 nL (height approx. 100 μm), the spot diameter is approx. 300 μm.

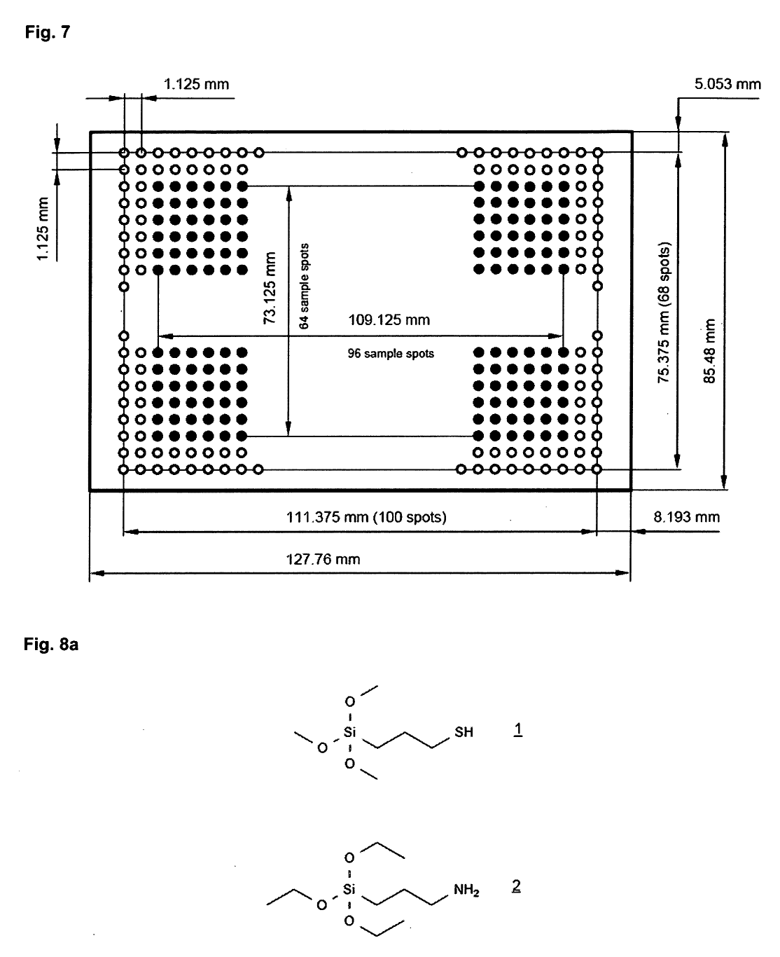

FIG. 7 shows a schematic drawing of the dimensions of an array according to a further preferred embodiment. The array footprint is 127.76 mm×85.48 mm. The thickness of the array is approx. 1.1 mm (standard) but 0.145 mm when used for immersion objective lenses requiring a close distance. The total number of spots is 100×68=6,800 spots. The outermost 2 spots of each row and column are not used for samples in order to avoid edge effects. The number of sample spots is 96×64=6,144 spots. The spot diameter is 0.600 mm; the spot to spot distance (center to center) is 1.125 mm. The density of the spots is 6,800 spots on 111.975 mm×75.975 mm=80 spots/cm². The sample volumes depend on the technique of liquid application. Usually, the volumes used are less than 55 nL (hemispheres). Typical volumes are about 35 nL (height approx. 200 μm), the spot diameter is approx. 600 μm.

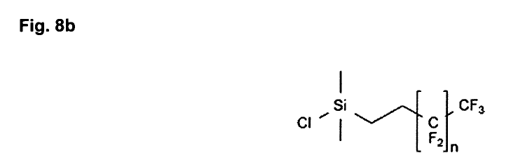

FIG. 8a shows the surface chemistry at the sample spots. The most favourable structures consist of 3-Aminopropyl-triethoxylsilane (1) or 3-Mercaptopropyl-trimethoxysilane (2), wherein (2) is most preferred. The monolayers are prepared by a CVD process at 100C° C.-150° C.

FIG. 8b shows the surface chemistry outside sample spots. The structures may consist of monolayers from chemisorption of 1 H, 1 H, 2H, 2H Perfluorodecyldimethylchlorosilane (n=7). Preferably, the structures consist of binary mixed monolayers of 1 H, 1 H, 2H, 2H Perfluoroalkyldimethylchlorosilanes with equimolar compositions of n=7 and n=19. Monolayers may be prepared by a CVD process at 100° C. to 150° C.

As is shown by the above explanation of the figures and preferred embodiments, the array has the subsequent functional specifications. The sample molecules are immobilized at sample spots, preferably chemically bonded to the sample spots. The cells are applied and adhere to sample spots. Due to the hydro- or ultraphobic area surrounding the sample spots, minimum non-specific binding of nucleic acids and constituents of cell culture media on areas outside sample spots occurs. Regarding the technical specifications, the sample spots of the array are designed for use in transmission light microscopy, that is, spots are optically transparent or translucent and provide optical properties similar to conventional microscopy slides. The sample plates are preferably compatible to instruments such as transmission optical microscopes and fluorescent readers.

1. A transfection method of introducing sample molecules into cells, comprising:
(a) using a substrate comprising at least one sample spot area which is surrounded by an hydrophobic area;
(b) wherein sample molecules are applied to said at least one sample spot area, thereby placing said sample molecules in the discrete location of said sample spot area;
(c) applying cells to be transfected onto the substrate under appropriate conditions for entry of the sample molecules into said cells;
(d) whereby at least a portion of said sample molecules are introduced into said cells.

2. The transfection method according to claim 1, wherein said sample spot area is wettable by the sample molecules.

3. The transfection method according to claim 2, wherein said sample spot area has hydrophilic and/or oleophilic properties.

4. The transfection method according to claim 1, wherein said hydrophobic area is hydrophobic and/ or oleophobic, preferably ultraphobic.

5. The transfection method according to claim 4, wherein said ultraphobic area depicts a contact angle in relation to water of at least 140°.

6. The transfection method according to claim 4, wherein said ultraphobic area is nano-structured.

7. The transfection method according to claim 6, wherein said ultraphobic area has a surface topography, having a topological frequency f of the individual Fourier components and wherein amplitude a(f) expressed by the integral S(log (f))=a(f)·f calculated between the integration limits log (f₁/μm⁻¹)=−3 and log (f₂/μm⁻¹)=3 is at least 0.3, and wherein the ultraphobic area is made of an hydrophobic or oleophobic material or is coated with a durable hydrophobic and/or durable oleophobic material.

8. The transfection method according to claim 1, wherein said sample molecules are covalently or non-covalently bound to said sample spot areas.

9. The transfection method according to claim 1, wherein said sample molecules are embedded in a matrix.

10. The transfection method according to claim 1, wherein a transfection reagent is applied to the substrate or is mixed with the cells to be transfected.

11. The transfection method according to claim 1, wherein at least said sample spot areas are optically transparent or translucent.

12. The transfection method according to claim 1, wherein the surface properties of said at least one sample spot area are modified to enhance the adherence of the transfection cells to the sample spot area.

13. The transfection method according to claim 1, wherein said substrate carries more than 1 sample spot areas, wherein said sample spot areas are arranged in an array format.

14. The transfection method according to claim 1, wherein at least two different kinds of sample molecules are applied to said at least one sample spot area.

15. The transfection method according to claim 1, wherein said sample molecule is selected from the group consisting of nucleic acids, peptides, small molecules, RNA molecule, RNAi mediating compound, and a siRNA molecule.

16. A transfection array for introducing sample molecules into cells, comprising a substrate comprising more than 1 sample spot areas arranged in an array format wherein said more than 1 sample spot areas are surrounded by a hydrophobic area and wherein said sample spot areas carry sample molecules useful for an transfection assay, whereby said sample molecules are located in the discrete location of said sample spot areas.

17. The transfection array according to claim 16, wherein the sample molecules comprise siRNA.

18. The transfection array according to claim 16, wherein said sample spot areas have hydrophilic and/or oleophilic properties.

19. The transfection array according to one of the claim 18, wherein the surface chemistry of the sample spot areas comprises a compound selected from the group consisting of 3-aminopropyl-triethoxysilane, 3 mercaptopropyl-trimethoxysilane, Methacryloxypropyl-triethoxysilane, Hexadecyltrimethoxysilane, 2-[Methoxy(polyethylenoxy)propyl]trimethoxysilane, 1 H, 1 H, 2H, 2H Perfluordecyltriethoxy-silane and 3-(phenylamino)propyltrimethyldiethoxysilane, preferably the compound is selected from 3-aminopropyl-triethoxysilane, 3 mercaptopropyl-trimethoxysilane and 3-(phenylamino)propyltrimethyldiethoxysilane.

20. The transfection array according claim 16, wherein said hydrophobic area is ultraphobic

21. The transfection array according to claim 20, wherein said ultraphobic area is nano-structured and depicts a contact angle in relation to water of at least 140°.

22. The transfection array according to claim 20, wherein said ultraphobic area has a surface topography, having a topological frequency f of the individual Fourier components wherein amplitudes a(f) expressed by the integral S(log (f))=a(f)·f calculated between the integration limits log (f₁/μm⁻¹)=−3 and log (f₂/μm⁻¹)=3 is at least 0.3, and wherein the ultraphobic area is made of an hydrophobic or oleophobic material or is coated with a durable hydrophobic and/or durable oleophobic material.

23. The transfection array according to claim 16, wherein at least said sample spot areas are optically transparent or translucent.

24. The transfection array according to claim 16, wherein at least some of the sample spot areas comprise a cell adherence promoting agent.

25. Use of a transfection array according to claim 16 in a transfection method.

26. Use of a transfection array according to claim 25, for performing a genome screening assay.
